(19) United States
(12) Patent Application Publication
(10) Pub. No.: US 2010/0168450 A1
(43) Pub. Date: Jul. 1, 2010

(54) NOVEL BRANCHED ALKOXYLATES

(76) Inventors: Johan A. Thoen, Antwerpen (BE); Camiel F. Bartelink, Terneuzen (NL); Pierre Varineau, Ankeny, IA (US)

Correspondence Address: The Dow Chemical Company, P.O. BOX 1967, Midland, MI, 48641, US

(21) Appl. No.: 12/596,919

(22) PCT Filed: Apr. 24, 2008
(86) PCT No.: PCT/US08/61365

§ 371 Date: Mar. 5, 2010

Related U.S. Application Data
(60) Provisional application No. 60/926,629, filed on Apr. 27, 2007.

(51) Int. Cl. C07D 301/00; C07D 303/18

(52) U.S. Cl. 549/539; 549/554

(57) ABSTRACT

Novel alkylene oxide-extended alkoxylates of a branched 1,3-dialkyl-oxy-2-propanol may be prepared by a convenient process comprising adding epichlorohydrin to a stoichiometric excess of a branched alcohol, wherein the molar ratio of branched alcohol:epichlorohydrin is at least about 3:1, preferably in the presence of a Group 1 A metal hydroxide and a phase transfer catalyst, followed by alkoxylation in the presence of an ionic catalyst. The branched alkyl chain may be saturated or unsaturated and may contain one or more heteroatoms. The repeating alkoxy units from the alkylene oxide are in the 2-position. The compositions are useful as surfactants, diluents, and the like, and may be less expensive than other branched surfactants.

### BACKGROUND OF THE INVENTION

1. Technical Field

This invention relates to the field of alkyloxy-ether alkoxylates. More particularly, it relates to compositions and processes for preparing branched alkyloxy-ether alkoxylates useful as surfactants.

2. Background of the Art

Surfactants are used in the chemical and manufacturing industries for a wide variety of purposes. These include, for example, imparting wettability and detergency in products including wetting agents, emulsifiers, rinse aids, defoam/low foam agents, spray cleaning agents, drug delivery agents, emulsifiers for herbicides and pesticides, metal cleaning agents, paints, coatings, agricultural spread and crop growth agents, stabilizing agents for latexes, paints and paper processing products, and the like. One group of frequently-employed surfactants is the nonionic surfactants. The nonionic surfactants tend to be generally less sensitive to hard water and generate less foam than some other types of surfactants, making many of nonionic surfactants useful as foam suppressants. Unfortunately, however, many of these surfactants in current use are alkylphenol-based compounds. Alkylphenol-based compounds have recently come under environmental scrutiny, and thus, compositions such as formulations and products containing them may eventually face restrictions.

One such alternative is the group of polyglycol ethers of higher saturated aliphatic monohydric alcohols. Etherification of glycerin was disclosed as early as 1959 in, for example, U.S. Pat. No. 2,870,220. Another method to prepare alkyl-ethers of glycerin is telomerization of the glycerin with 1,4-butadiene, followed by hydrogenation, as described in, for example, A. Behr, M. Urschey, “Highly Selective Biphasic Telomerization of Butadiene with Glycols: Scope and Limitations,” Adv. Synth. Catal. 2003, 345, 1242-1246; DE 10105751 A1 (2002); and DE10128144 A1 (2002). Both of these methods tend to result in mixtures of 1-, 2-, and 3-substituted glycerins. Another U.S. publication, US2002/0004605 A1 (2002), describes a process for making 1,3-dioctyloxy-2-propanol without the addition of a solvent, by reacting fatty alcohols with epichlorohydrin in the presence of an alkali metal hydroxide and a phase transfer catalyst in specified molar ratios. This method, however, is characterized by relatively low selectivity of the 1,3-substitution product due to the formation of heavy by-products, such as 14-(octyloxymethyl)-9,13,16-trioxa-tetracosan-11-ol.

Other methods of preparing nonionic surfactants known in the art include ethoxylation of higher aliphatic secondary alcohols in the presence of an acidic catalyst, the product then being further ethoxylated in the presence of an alkaline catalyst to produce products with multiple moles of ethylene oxide per mole of alcohol. See, e.g., EP 0 043 963 A1 (1982). A combination of ethylene oxide and propylene oxide may alternatively be used for the second ethoxylation, the result thereof being a block copolymer. These copolymers may be particularly useful as surfactants in processes where they are exposed to mechanical agitation and heat. However, the performance of many of these products may not, in some cases, be as good as that of the alkylphenol-based surfactants.

Thus, there is a need in the art to identify compositions and processes for surfactants that provide performance that is at least comparable to the alkylphenol ethoxylates at an attractive cost.

### SUMMARY OF THE INVENTION

Accordingly, the present invention provides, in one aspect, a process for preparing a branched surfactant comprising reacting 1-chloro-2,3-epoxypropane and a stoichiometric excess of a branched alcohol, such that the molar ratio of branched alcohol to 1-chloro-2,3-epoxypropane is at least about 3:1 during the reaction, to form a reaction product, and reacting the reaction product with from 2 to 12 moles of ethylene oxide per mole of reaction product in the presence of an ionic catalyst, to form a branched 1,3-dialkyloxy-2-propanol. The branched alcohol starting material may be saturated or unsaturated and optionally contains one or more heteroatoms selected from the group consisting of elements of Groups IVA, VA, VIA and VIIA of the Periodic Table and combinations thereof. The alcohol is, in non-limiting embodiments, selected from C3 to C28 methyl-, ethyl-, propyl-, and butyl-branched aliphatic alcohols.

### DETAILED DESCRIPTION OF THE INVENTION

The inventive process for preparing a branched, ethylene-oxide extended dialkyl-ether of glycerin offers the possibility of advantageously economical commercial production. The starting materials include, first, epichlorohydrin, also termed 1-chloro-2,3-epoxypropane. Those skilled in the art will be aware of a large number of commercial sources for this material, which may be generally prepared by the reaction of propylene and an allyl chloride, or, for instance, by the conversion of a multihydroxylated-aliphatic hydrocarbon or ester thereof to a chlorohydrin, such as is described in WO 2006020234 A1, the disclosure of which is incorporated herein by reference in its entirety.

The second starting material is a branched alcohol. This branched alcohol, in some non-limiting embodiments, has from 3 to 28 carbon atoms, and in other non-limiting embodiments, has from 4 to 12 carbon atoms. In particularly preferred embodiments the alkyl chain may include from 6 to 10 carbon atoms. The branched alcohol may be a primary, secondary or tertiary branched alcohol; may be saturated or unsaturated; and may optionally contain one or more heteroatoms. For example, in certain non-limiting embodiments appropriate selections may include renewable feedstocks, such as bio-glycerin; 2-ethylhexanol; certain NEODOL™ branched alcohols marketed by Shell Chemical Company; certain EXXAL™ branched alcohols marketed by Exxon-Mobil Corporation; combinations thereof; and the like. In other non-limiting embodiments, suitable alcohols may include, for example, 4-propyl-1-decanol, 3-butyl-1-nonanol, 2-methyl-4-propyl-1-decanol, 3-methyl-1-heptanol, 3-methyl-2-heptanol, 3-methyl-3-heptanol, 2-ethyl-1-hexanol, 2-methyl-1-heptanol, 2-methyl-1-pentanol, combinations thereof, and the like. Thus, in certain non-limiting embodiments the alcohol may be selected such that it contains as its alkyl moiety a moiety selected from the group consisting of methyl-, ethyl-, propyl- and butyl-branched pentyl-, hexyl-, heptyl-, octyl-, nonyl-, decyl-, undecyl-, dodecyl- and tridecyl-aliphatic moieties.

The branched alcohol may contain, as heteroatoms, elements selected from Groups IVA, VA, VIA and VIIA of the Periodic Table of the Elements, including, but not limited to, elements such as sulfur, phosphorus, and silicon; non-metals such as nitrogen, fluorine and oxygen; combinations thereof; and the like. In certain non-limiting embodiments the branched alcohol may be, for example, a branched heptanol, or a branched alcohol produced according to methods such as those described in WO 2003024910 A1, assigned to Sasol Tech PTY LTD, the disclosure of which is incorporated herein by reference in its entirety. For example, in certain non-limiting embodiments, 2-methylpentanol or 2-methyl-heptanol may be selected.

The first step in the process is to react the epichlorohydrin with an excess of the branched alcohol. Such requires addition of the epichlorohydrin in any manner in which the desired stoichiometric excess may be maintained. For example, on a large or commercial scale, the epichlorohydrin may be added continuously. In contrast, on a smaller scale (e.g., laboratory scale), a “stepwise” manner may be more conveniently employed. This may comprise adding an amount of the epichlorohydrin in each of at least three steps, and in some non-limiting embodiments, in each of at least five steps. Time between steps may be varied, provided that the desired excess of branched alcohol is maintained throughout the reaction. In certain non-limiting embodiments it may be from about 30 minutes to about 90 minutes; in other non-limiting embodiments it may be from about 45 minutes to about 75 minutes; and in still other non-limiting embodiments it may be about 60 minutes. The stepwise addition may be particularly helpful in controlling the exotherm for such small-scale reactions.

The stoichiometric excess is defined herein as meaning that, at all times throughout the reaction, the branched alcohol is present in the reaction in an amount that is at least three times the stoichiometric amount based on the epichlorohydrin, i.e., the branched alcohol:epichlorohydrin molar ratio is at least about 3:1. However, it has been found useful in some embodiments to begin with a much greater excess of the branched alcohol, such as from about 10:1 to about 20:1, and then to increase the relative amount or rate of addition of epichlorohydrin until, toward the end of the reaction, there is approximately a 3:1 alcohol:epichlorohydrin ratio. In other non-limiting embodiments, successful reactions may be carried out by maintaining ratios of from about 15:1 to about 16:1 throughout most of the reaction, whether the epichlorohydrin is being added stepwise or continuously, and then increasing the amount or rate of addition of epichlorohydrin toward the end of the reaction such that the ratio of alcohol:epichlorohydrin drops to about 3:1. In addition to aiding exotherm control, employing such a controlled protocol in incorporating the epichlorohydrin into the reaction may assist in reducing the amount of so-called heavies. These heavies, which result from further reaction of the alkyloxy-ether, are impurities in the end product that have a boiling point that is higher than that of the desired alkyloxy-ether.

This reaction also desirably includes the presence of an alkaline environment and a phase transfer catalyst. The alkaline environment may be obtained by addition of a metal hydroxide, including a Group 1A metal, for example, sodium hydroxide or potassium hydroxide. In certain non-limiting embodiments the metal hydroxide is combined with the branched alcohol prior to addition of the epichlorohydrin, while in other, though less preferred, embodiments, the metal hydroxide and branched alcohol may be combined simultaneously with the epichlorohydrin.

Overall molar proportions of the alcohol, metal hydroxide and epichlorohydrin may range, and/or be varied, in certain non-limiting embodiments, from about 1/0.7/0.06 to a final molar ratio of from about 1/0.7/0.2 to 1/0.7/0.33, and, in a particular embodiment, to about 1/0.7/0.3. In other non-limiting embodiments, the proportion of alcohol/metal hydroxide/epichlorohydrin, either immediately following each addition of the epichlorohydrin where such is done stepwise, or in continuous productions, throughout most of the duration of the reaction, may range from about 1/0.7/0.01 to about 1/0.7/0.08, preferably from about 1/0.7/0.02 to about 1/0.7/0.1, and more preferably from about 1/0.7/0.05 to about 1/0.7/0.07. In certain non-limiting embodiments this ratio may be ramped up, toward the end of the reaction, to range from about 1/0.7/0.2 to 1/0.7/0.33, preferably about 1/0.7/0.33.

The phase transfer catalyst used for the reaction between the branched alcohol and the epichlorohydrin may be selected from those typically known to those skilled in the art. For example, those that may be selected include salts having anions selected from the group consisting of halide, methylsulfate, and hydrogensulfate, such as alkyldimethylbenzylammonium salt, tetraalkylammonium salt, N,N,N-trialky-3-alkyloxy-2-hydroxypropylammonium salt and alkyltrimethyl-ammonium salt. Other examples include trialkylamine, N,N-dialkylamino-3-alkyloxy-2-propanol, tetrabutylammonium bromide, tetrabutylammonium hydrogensulfate, cetyltrimethylammonium chloride, lauryldimethylbenzyl-ammonium chloride, N,N-dimethylamino-3-hexyloxy-2-propanol, N,N-dimethyl-amino-3-octyloxy-2-propanol, N,N-dimethylamino-3-dodecyloxy-2-propanol, N,N-dimethylamino-3-octadecyloxy-2-propanol, N,N-dimethylamino-3-(1′H,1′H,2′H,2′H-perfluoro)hexyloxy-2-propanol, N,N-dimethyl-amino-3-(1′H,1′H,2′H,2′H-perfluoro)-octyloxy-2-propanol, N,N-bis(2-hydroxyethyl)-amino-3-hexyloxy-2-propanol, N,N-bis(2-hydroxyethyl)amino-3-octyloxy-2-propanol, N,N-bis(2-hydroxyethyl)amino-3-dodecyloxy-2-propanol, N,N-bis(2-hydroxyethyl)amino-3-octadecyloxy-2-propanol, N,N-bis(2-hydroxyethyl)-amino-3-1′H,1′H,2′H,2′H-perfluoro)hexyloxy-2-propanol, N,N-bis(2-hydroxypropyl-ammonium methylsulfate, N,N,N-trimethyl-3-octyloxy-2-hydroxypropylammonium methylsulfate, N,N,N-trimethyl-3-dodecyloxy-2-hydroxy-propylammonium methyl-sulfate, N,N,N-trimethyl-3-octyloxy-2-hydroxypropyl-ammonium chloride, N,N,N-trimethyl-3-octyloxy-2-hydroxypropylammonium bromide, N,N-bis(2-hydroxyethyl)-N-methyl-3-hexyloxy-2-hydroxypropylammonium methylsulfate, N,N-bis(2-hydroxy-ethyl)-N-methyl-3-octyloxy-2-hydroxypropyl-ammonium methylsulfate, N,N-bis(2-hydroxyethyl)-N-methyl-3-dodecyloxy-2-hydroxypropylammonnium methylsulfate, N, N-bis(2-hydroxyethyl)-N-methyl-3-octadecyloxy-2-hydroxpropyl ammonium methylsulfate, N,N-bis(2-hydroxyethyl)-N-methyl-3-(1′H,1′H,2′H,2′ H-perfluoro)-hexyloxy-2-hydroxy-propylammonium methyl-sulfate, N,N-bis(2-hydroxyethyl)-N-methyl-3-(1′H,1′H,2′H,2′H-perfluoro)-octyloxy-2-hydroxypropylammonium methyl-sulfate, an esterified compound of octanoic acid and N,N-dimethyl-3-oxtyloxy-2-propanol, an esterified compound of hexadecanoic aid and N,N-dimetyl-3-octyloxy-2-propanol, and the like. Combinations of any of the above may alternatively be selected.

The reaction of the epichlorohydrin and branched alcohol is desirably carried out at a temperature of from about 10° C. to about 100° C. and a pressure of from about 1 atmosphere (atm) to about 10 atm, i.e., about 760-7600 Torr. Appropriate mixing of the reactants to maximize contact thereof is desirable upon, and during, each addition of the epichlorohydrin. Such may be accomplished by any means or method known to those skilled in the art, such as, for example, an impeller mixer, a blade mixer, a recirculation mixer, or the like.

The result of the reaction is formation of a reaction product. This reaction product may, in certain non-limiting embodiments, be primarily a dialkyl-ether of the selected branched alcohol, with good selectivity at the 1- and 3-positions. In other non-limiting embodiments, the branched 1,3-dialkyl-ether may be at least about 50 percent; in other non-limiting embodiments, the branched 1,3-dialkyl-ether may be at least about 65 percent; and in still other non-limiting embodiments, the branched 1,3-dialkyl-ether may be at least about 75 percent; all based on the weight of the reaction product, i.e., not including the unreacted branched alcohol.

In a second step, the reaction product obtained as described hereinabove is then reacted with an alkylene oxide to form a 1,3-dialkyl-ether alkoxylate. Suitable alkylene oxides are any having, in certain non-limiting embodiments, from 2 to 12 carbon atoms. These include, for example, ethylene oxide, propylene oxide, butylene oxide, and the like. In certain embodiments, ethylene oxide may be selected. In other non-limiting embodiments, propylene oxide may be selected, and in still other non-limiting embodiments, a mixture of ethylene oxide and propylene oxide may be selected. Where a mixture is used, the result is a copolymer.

This second alkoxylation, to form the 1,3-dialkyl-ether alkoxylate, is desirably carried out in the presence of at least one ionic catalyst. In one particularly desirably embodiment, at least two ionic catalysts are used, in sequence, with a cationic catalyst employed during the addition of the first few moles of alkylene oxide, and then an anionic catalyst used during the addition of the desired remainder of the alkylene oxide. Alternatively, a single ionic catalyst, or single type of ionic catalyst (i.e., either cationic or anionic), may be used throughout the second alkoxylation.

Possible selections for a cationic catalyst may include acidic catalysts, i.e., cationic polymerization catalysts, such as those known as Friedel-Crafts type reaction catalysts. Such may include, for example, fluorides and chlorides of boron, aluminum, iron, tin and titanium, and complexes of such halides with ethyl ether. In one embodiment, boron trifluoride may be selected. In another embodiment, trifluoromethane sulfonic acid may be selected. In still other embodiments, sulfuric acid or phosphoric acid may be selected. Combinations of any of the above may also be used.

Possible selections for an anionic catalyst may include alkaline catalysts, i.e., anionic polymerization catalysts, such as Group 1A metal hydroxides, for example, potassium hydroxide. Alkali metal alcoholates, for example, of the initial branched alcohol, or the corresponding alcoholate of the 1,3-dialkyloxy-2-propanol made during the first stage of the process, may also be selected. Such catalysts may be made in situ by reacting the neutralized product of the first reaction stage with an alkali metal, alkali metal oxide or hydroxide, or may be obtained as neat compositions. Combinations of anionic catalysts may also be selected.

The proportion of the branched 1,3-dialkyl-ether, i.e., the 1,3-dialkyloxy-2-propanol, to the alkylene oxide may range as a molar ratio of from about 1:2 to about 1:20. In certain non-limiting embodiments this ratio may be from about 1:3 to about 1:15, and in other non-limiting embodiments it may range from about 1:5 to about 1:12.

Both the branched 1,3-dialkyloxy-ether and the branched 1,3-dialkyloxy-ether alkoxylate may exhibit utility as surfactants, reactive diluents in casting, encapsulation, flooring, potting, adhesives, laminating, reinforced plastics, filament windings, coating and electrotechnical applications such as encapsulating of magnet coils and high voltage coils, wetting agents, emulsifiers, rinse aids, defoam/low foam agents, spray cleaning agents, drug delivery agents, emulsifiers for herbicides and pesticides, metal cleaning agents, paints, coatings, agricultural spread and crop growth agents, stabilizing agents for latexes, paints and paper processing products, and the like. and the like. For example, the surfactants described herein may serve to dilute higher viscosity epoxy resins based on bisphenol-A, bisphenol-F, and novolak, as well as other polymers such as polyurethanes and acrylics. They may also find use in rheology modification of liquid systems such as inks, emulsions, paints, and pigment suspensions, where they may be used to impart pseudoplasticity or thixotropic flow behavior. In these and other uses they may offer good and, in some cases, excellent performance, as well as relatively low cost.

It is commonly known to those skilled in the art that levels of surfactant in such applications may range from about 0.05 to about 50 weight percent, more frequently from about 0.1 to about 30 weight percent, and in some uses from about 0.5 to about 20 weight percent. Those skilled in the art will be able to determine usage amounts via a combination of general knowledge of the applicable field as well as routine experimentation where needed.

The description hereinabove is intended to be general and is not intended to be inclusive of all possible embodiments of the invention. Similarly, the examples hereinbelow are provided to be illustrative only and are not intended to define or limit the invention in any way. Those skilled in the art will be aware that the unreacted branched alcohol may be recovered, dried, and recycled using means and methods that are well-known. Appropriate distillation systems may be employed in order to improve product quality, and such may be carried out continuously, particularly if the reaction system or systems is/are set up for continuous operation. Finally, the reaction may be set up such that the intermediates, e.g., 1-(2-methylheptyloxy)-3-chloro-2-propanool and/or 2-methyl-heptyl glycidyl ether, are either reduced to acceptable levels, by continuing the reaction to a desired point, or by recovering and/or recycling the intermediates. Such process variations further reduce the costs of the process and efficiency thereof.

Furthermore, those skilled in the art will be fully aware that other embodiments within the scope of the claims will be apparent, from consideration of the specification and/or practice of the invention as disclosed herein. Such other embodiments may include selections of specific branched alcohols, catalysts, and combinations of such compounds; proportions of such compounds; mixing and reaction conditions, vessels, and protocols; performance and selectivity; applications of the final branched, alkylene-oxide extended dialkyl-ether alkoxylate; and the like; and those skilled in the art will recognize that such may be varied within the scope of the appended claims hereto.

### EXAMPLES

### Example 1

A. Preparation of a Branched 1,3-dialkyloxy-2-propanol

To a 1-liter bottle in a film hood, without rigorous exclusion of air or moisture, is added about 24.4 g of tetrabutylammonium bromide, about 66.4 g of sodium hydroxide that has been ground to a fine powder, and about 305.7 g of 2-methyl-1-heptanol. The bottle is shaken to dissolve the tetrabutylammonium bromide. About 13.1 g of 1-chloro-2,3-epoxypropane is then added and the bottle is shaken to mix the suspension.

After one hour about 13.1 g of 1-chloro-2,3-epoxypropane is added again and the bottle is shaken to mix the suspension. Three more additions of 1-chloro-2,3-epoxypropane are carried out at one-hour intervals, for a total of five such additions, totaling 65.4 g of 1-chloro-2,3-epoxypropane. The reaction is monitored by gas chromatography until all of the 1-chloro-2,3-epoxypropane is consumed.

The reaction product is then analyzed to contain a proportion of 1,3-di-(2-methyl heptyloxy)-2-propanol.

The reaction described herein is repeated 16 times. The combined batches are then filtered through a coarse sintered glass funnel to remove salt and unreacted sodium hydroxide and the filtrate is washed with deionized water. Light fractions, primarily 2-methyl-1-heptanol, are removed by stripping on a rotary evaporator with a heating bath set at 90° C., by lowering the pressure at a rate to prevent bumping until a final pressure of about 0.5 mm is reached. The stripped material has a higher proportion of the 1,3-di-(2-methylheptyloxy)-2-propanol.

B. Separation of the 1,3-di-(2-methylheptyloxy)-2-propanol

The stripped material is distilled in a batch distillation apparatus consisting of a 2-liter kettle heated with a heating mantle, magnetic stirring, a thermowell, and a one-piece distilling head/condenser. The distillation is conducted by reducing the pressure to full vacuum pump pressure (0.2 to 0.5 mm) and slowly increasing the mantle temperature. Cuts taken below an overhead temperature of 155° C. contains light fractions such as 2-methyl-1-heptanol and 1-(2-methylheptyloxy)-3-chloro-2-propanol. The 1,3-di-(2-methylheptyloxy)-2-prop-anol is the overhead product when the overhead temperature is from about 138-160° C. and the kettle temperature is below 200° C.

C. Further Separation of the 1,3-di-(2-methylheptyloxy)-2-propanol

The initial distillation in part “B” hereinabove results in a product including both 1,3-di-(2-methylheptyloxy)-2-propanol and the undesirable contaminant, 1-(2-methylheptyloxy)-3-chloro-2-propanol. A strong base is added to the distillation product in an attempt to convert the 1-(2-methylheptyloxy)-3-chloro-2-propanol to 2-methylheptyl glycidyl ether, which is expected to react further to form the high boiling compound. The cuts from the first distillation, contaminated with 1-(2-methylheptyloxy)-3-chloro-2-propanol, are combined with the remaining stripped material and treated with sodium hydride. The sodium hydride is a 60 percent by weight solution in mineral oil as received from a commercial producer, but the weight of this initial charge, recorded as the weight of the crude 1,3-di-(2-methylheptyloxy)-2-propanol solution, does not include the mineral oil. This initial charge is roughly estimated to be equimolar to the chlorohydrin concentration and results in a reduction of the 1-(2-methylheptyloxy)-3-chloro-2-propanol concentration. Repeating the sodium hydride treatment reduces the 1-(2-methylheptyloxy)-3-chloro-2-propanol concentration further. The resulting hydride material is washed with dilute HCl followed by a wash with saturated sodium carbonate.

The crude washed material is then subjected to another batch distillation as described hereinabove. Cuts are collected when the overhead temperature is from about 138-160° C. and are combined to give a relatively high proportion of 1,3-di-(2-methylheptyloxy)-2-propanol.

D. Preparation of the Branched ether-alkoxylate (ethoxylation of 1,3-di-(2-methylheptyloxy)-2-propanol)

In separate reactions, five samples of the purified 1,3-di-(2-methylheptyloxy)-2-propanol are individually reacted with varying molar amounts of ethylene oxide, using potassium hydroxide (KOH), trifluoromethane sulfonic acid (CF₃SO₃H), or boron trifluoride (BF₃) as the catalyst. The molar amounts are 2, 6, 9 and 12, with two samples prepared using 2 moles of ethylene oxide each.

1. A process for preparing a branched surfactant comprising
reacting 1-chloro-2,3-expoxypropane and a stoichiometric excess of a branched alcohol, such that the molar ratio of the branched alcohol to 1-chloro-2,3-epoxypropane is at least about 3:1 during the reaction, to form a reaction product, and reacting the reaction product with from 2 to 12 moles of an alkylene oxide per mole of reaction product in the presence of an ionic catalyst, to form a branched, alkylene oxide-extended 1,3-dialkyloxy-2 propanol alkoxylate.

2. The process of claim 1 wherein the branched alcohol has from 3 to 28 carbon atoms, and is saturated or unsaturated.

3. The process of claim 1 wherein the branched alcohol contains at least one heteroatom selected from the group consisting of elements of Group IVA, VA, VIA and VIIA, and combinations thereof.

4. The process of claim 1 wherein the 1-chloro-2,3-epoxypropane is added to the branched alcohol continuously or stepwise.

5. (canceled)

6. The process of claim 1 wherein the molar ratio of branched alcohol/metal hydroxide/1-chloro-2,3-epoxypropane during the reaction is from about 1/0.7/0.01 to about 1/0.7/0.10.

7. The process of claim 6 wherein the molar ratio of branched alcohol/metal hydroxide/1-chloro-2,3-epoxypropane during the reaction is from about 1/0.7/0.06 to about 1/0.7/0.3.

8. The process of claim 1 wherein the molar ratio of branched alcohol/metal hydroxide/1-chloro-2,3-epoxypropane is varied during the reaction, from about 1/0.7/0.01 to about 1/0.7/0.33.

9. The process of claim 1 further comprising a phase transfer catalyst.

10. The process of claim 9 wherein the phase transfer catalyst is selected from the group consisting of trialkylamine, alkyldimethylbenzylammonium salt, tetraalkylammonium salt, N,N-dialkylamino-3-alkyloxy-2-propanol, N,N,N-trialkyl-3-alkyloxy-2-hydroxypropyl-ammonium salt and alkyltrimethylammonium salt, wherein the salt includes an anion selected from the group consisting of halide, methylsulfate, and hydrogensulfate; tetrabutylammonium bromide, tetrabutylammonium hydrogensulfate, cetyltrimethylammonium chloride, lauryldimethylbenzylammonium chloride, N,N-dimethylamino-3-hexyloxy-2-propanol, N,N-dimethylamino-3-octyloxy-2-propanol, N,N-dimethylamino-3-dodecyloxy-2-propanol, N,N-dimethylamino-3-octadecyloxy-2-propanol, N,N-dimethylamino-3-(1′H,1′H,2′H,2′H-perfluoro)hexyloxy-2-propanol, N,N-dimethyl-amino-3-(1′H,1′H,2′H,2′H-perfluoro)octyloxy-2-propanol, N,N-bis(2-hydroxyethyl)-amino-3-hexyloxy-2-propanol, N,N-bis(2-hydroxyethyl)amino-3-octyloxy-2-propanol, N,N-bis(2-hydroxyethyl)amino-3-dodecyloxy-2-propanol, N,N-bis(2-hydroxyethyl)amino-3-octadecyloxy-2-propanol, N,N-bis(2-hydroxyethyl)-amino-3-1′H,1′H,2 ′H,2′H-perfluoro)hexyloxy-2-propanol, N,N-bis(2-hydroxypropyl-ammonium methylsulfate, N,N,N-trimethyl-3-octyloxy-2-hydroxypropylammonium methyl-sulfate, N,N,N-trimethyl-3-dodecyloxy-2-hydroxypropylammonium methyl-sulfate, N,N,N-trimethyl-3-octyloxy-2-hydroxypropylammonium chloride, N,N,N-trimethyl-3-octyloxy-2-hydroxypropylammonium bromide, N,N-bis(2-hydroxyethyl)-N-methyl-3-hexyloxy-2-hydroxypropylammonium methylsulfate, N,N-bis(2-hydroxy-ethyl)-N-methyl-3-octyloxy-2-hydroxypropylammonium methylsulfate, N,N-bis(2-hydroxy-ethyl)-N-methyl-3-dodecyloxy-2-hydroxypropylammonnium methylsulfate, N,N-bis(2-hydroxyethyl)-N-methyl-3-octadecyloxy-2-hydroxpropylammonium methyl-sulfate, N,N-bis(2-hydroxyethyl)-N-methyl-3-(1′H,1′H,2′H,2′H-perfluoro)-hexyloxy-2-hydroxy-propylammonium methylsulfate, N,N-bis(2-hydroxyethyl)-N-methyl-3-(1′H,1′H,2′H,2′H-perfluoro)octyloxy-2-hydroxypropylammonium methyl-sulfate, an esterified compound of octanoic acid and N,N-dimethyl-3-oxtyloxy-2-propanol, an esterified compound of hexadecanoic aid and N,N-dimetyl-3-octyloxy-2-propanol, and combinations thereof.

11. The process of claim 1 wherein the reacting is carried out at a temperature from about 10° C. to about 100° C.

12. The process of claim 1 wherein the molar ratio of the branched 1,3-dialkyloxy-2-propanol to alkylene oxide is from 1:2 to 1:20.

13. The process of claim 12 wherein the molar ratio of the branched 1,3-dialkyloxy-2-propanol to alkylene oxide is from about 1:3 to about 1:15.

14. The process of claim 1 wherein the branched 1,3-dialkyloxy-2-propanol alkoxylate contains an alkyl moiety selected from the group consisting of methyl-, ethyl-, propyl- and butyl-branched pentyl-, hexyl-, heptyl-, octyl-, nonyl-, decyl-, undecyl-, dodecyl- and tridecyl-aliphatic moieties.

15. The process of claim 12 wherein the branched 1,3-dialkyloxy-2-propanol alkoxylate exhibits surfactant properties.

16. A surfactant composition comprising a branched 1,3-dialkyloxy-2-propanol alkoxylate prepared by a process comprising reacting a 1-chloro-2,3-epoxypropane and a stoichiometric excess of a branched alcohol having from 3 to 28 carbon atoms, such that the molar ratio of branched alcohol to 1-chloro-2,3-epoxypropane is at least about 3:1 during the reaction, in the presence of a metal hydroxide and a phase transfer catalyst, such that a reaction product comprising a branched 1,3-dialkyloxy-2-propanol alkoxylate is formed; and reacting the reaction product and from about 2 to about 12 moles of an alkylene oxide per mole of reaction product, in the presence of an ionic catalyst, to form a branched, alkylene oxide-extended 1,3-dialkyloxy-2-propanol alkoxylate.

17. The surfactant composition of claim 16 wherein the reaction product comprises the branched 1,3-dialkyloxy-2-propanol in an amount of at least about 65 percent by weight.

18. The surfactant composition of claim 16 wherein the yield of branched 1,3-dialkyloxy-3-propanol is at least about 50 percent of theoretical based on the 1-chloro-2,3-epoxypropane.

19. The surfactant composition of claim 16 wherein the branched 1,3-dialkyloxy-2-propanol is in a molar ratio relative to the alkylene oxide from 1:2 to 1:20.

20. The surfactant composition of claim 19 wherein the branched 1,3-dialkyloxy-2-propanol is in a molar ratio relative to the alkylene oxide from 1:3 to 1:15.

21. The surfactant composition of claim 16 wherein the ionic catalyst is selected from the group consisting of cationic catalysts, anionic catalysts, and combinations thereof.
